(19) United States
(12) Patent Application Publication
(10) Pub. No.: US 2023/0181085 A1
(43) Pub. Date: Jun. 15, 2023

(54) METHOD FOR DETECTING AN ECTOPIC SIGNAL IN AN ELECTROCARDIOGRAM

(71) Applicant: BIOTRONIK SE & Co. KG, Berlin (DE)

(72) Inventors: Garth Garner, Tigard, OR (US); Iris Shelly, Portland, OR (US); R. Hollis Whittington, Portland, OR (US)

(73) Assignee: BIOTRONIK SE & Co. KG, Berlin (DE)

(21) Appl. No.: 17/921,159

(22) PCT Filed: Apr. 20, 2021
(86) PCT No.: PCT/EP2021/060174

§ 371 Date: Oct. 25, 2022

Related U.S. Application Data
(60) Provisional application No. 63/017,709, filed on Apr. 30, 2020.

(30) Foreign Application Priority Data
Jul. 1, 2020 (EP) 20183409.0

(51) Int. Cl. A61B 5/364; A61B 5/352; A61B 5/361

(52) U.S. Cl. CPC A61B 5/364; A61B 5/352; A61B 5/361

(57) ABSTRACT

A method for detecting an ectopic signal in an electrocardiogram is disclosed. This method comprises the following steps: detecting consecutive R-R intervals in an electrocardiogram; calculating an average R-R interval for a determinable number of latest R-R intervals; recognizing a signal as ectopic signal if the signal belongs to at least one of two consecutive R-R intervals, wherein i) a first of the two consecutive R-R intervals is significantly shorter than the average R-R interval; and ii) a second of the two consecutive R-R intervals is significantly longer than the average R-R interval, wherein the second R-R interval occurs later than the first R-R interval; wherein the first and the second of the two consecutive R-R intervals are discarded from calculating the average R-R interval, if the signal is recognized as ectopic signal.

### CROSS-REFERENCE TO RELATED APPLICATIONS

This application is the United States National Phase under 35 U.S.C. § 371 of PCT International Patent Application No. PCT/EP2021/060174, filed on Apr. 20, 2021, which claims the benefit of European Patent Application No. 20183409.0, filed on Jul. 1, 2020, and U.S. Provisional Patent Application No. 63/017,709, filed on Apr. 30, 2020, the disclosures of which are hereby incorporated by reference herein in their entireties.

### TECHNICAL FIELD

The present invention relates in an aspect generally to a method for detecting an ectopic signal in an electrocardiogram. In another aspect, the present invention relates to an implantable medical device for detecting an electrical signal of a human or animal heart. In yet another aspect, the present invention relates to a method for detecting atrial fibrillation of a human or animal heart.

### BACKGROUND

Implantable medical devices for detecting atrial fibrillation have to work reliably so as to be able to detect real atrial fibrillation but to not falsely detect atrial fibrillation. However, ectopic cardiac beats resulting in ectopic signals in an electrocardiogram (also referred to as ectopies) often distort atrial fibrillation detection. Thus, there is a need in reliably detecting ectopic events in an electrocardiogram.

Ectopies can be caused by premature atrial contractions (PACs) and by premature ventricular contractions (PVCs). PACs and PVCs are typically preceded with a short interval followed by a long compensatory pulse interval. PACs typically lead to the same QRS complex morphology like a regular cardiac rhythm. This is due to the fact that the electrical conduction to the ventricle makes use of the normal cardiac conduction pathway.

In contrast, PVCs typically have a different ventricular morphology because of a different electrical conduction pathway. Algorithms known from prior art detecting ectopies (caused by PACs or PVCs) typically include interval timing looking for short-long intervals.

U.S. Publication No. 2013/0218037 A1 discloses a method for removal of ectopic beats by computing a ratio between an R-R interval comprising an ectopic signal and a preceding (presumably normal) R-R interval. Alternatively, an R-R interval comprising an ectopic beat is compared with a preceding and a succeeding R-R interval, wherein at the same time an R-R interval succeeding an R-R interval comprising an ectopic beat is compared to this R-R interval comprising an ectopic beat as well as to the next successive R-R interval.

U.S. Pat. No. 8,977,350 B2 discloses a method for identifying ectopic beats by comparing R-R interval lengths of consecutive R-R intervals.

The present disclosure is directed toward overcoming one or more of the above-mentioned problems, though not necessarily limited to embodiments that do.

### SUMMARY

It is an object of the present invention to provide a method that allows detection of ectopic signals in an electrocardiogram more reliably than the methods known from prior art.

At least this object is achieved, in an aspect, by a method for detecting an ectopic signal in an electrocardiogram comprising the steps explained in the following. In this context, the ectopic signal is caused by an ectopic cardiac beat.

In a first step, consecutive R-R intervals are detected or identified in an electrocardiogram.

Afterwards, an average R-R interval is calculated for a determinable number of latest R-R intervals. To give an example, the determinable number of latest R-R intervals can be 1, 2, 3, 4, 5, 6, 7, 8, 9, or 10. According to an embodiment, the latest R-R intervals considered are the latest consecutive intervals. In an embodiment, the determinable number of latest consecutive R-R intervals is a number lying in a range between 2 and 20, in particular between 3 and 19, in particular between 4 and 18, in particular between 5 and 17, in particular between 6 and 16, in particular between 7 and 15, in particular between 8 and 14, in particular between 9 and 13, in particular between 10 and 12.

Afterwards, the signal is recognized or identified as ectopic signal if the signal belongs to at least one of two consecutive R-R intervals and if the two conditions explained in the following are fulfilled. In this context, it should be noted that a signal typically belongs to two different consecutive R-R intervals since the first R-R interval ends at the signal, wherein a consecutive second R-R interval starts at the same signal. However, in certain instances, it might also be possible that an individual cardiac signal only belongs to one R-R interval.

The following two conditions need to be met for recognizing a signal as an ectopic signal. First, the first of the two consecutive R-R intervals needs to be at least 5% shorter than the average R-R interval. Second, a second of the two consecutive R-R intervals needs to be at least 5% longer than the average R-R interval. In this context, the second R-R interval occurs later than the first R-R interval in the electrocardiogram.

In order to achieve a particularly reliable recognition of ectopic signals, the first and the second of the two consecutive R-R intervals are discarded from calculating the average R-R interval if the signal is recognized as ectopic signal. Avoiding using of the two consecutive R-R intervals (also referred to as ectopic R-R intervals) for calculating the average R-R interval results in a more reliable average R-R interval and thus in a more reliable detection of subsequent ectopic signals.

In contrast to advanced computational methods used by many prior art ectopy detection algorithms, the presently described method can be performed with only very low power consumption so that it is particularly appropriate to be used in low-power implants.

In an embodiment, the method further comprises the steps explained in the following. A first deviation is calculated, wherein the first deviation is an average absolute deviation of the consecutive R-R intervals from the average R-R interval. In this context, the consecutive R-R intervals belong to the determinable number of latest consecutive R-R intervals used for calculating the average R-R interval. This first deviation is then multiplied with a factor being greater than one, resulting an increased first deviation. Furthermore, a second deviation is calculated. The second deviation is an absolute deviation of the successive R-R interval from the average R-R interval. “Successive R-R interval” means that this R-R interval succeeds the latest R-R interval of the determinable number of latest consecutive R-R intervals. The considered R-R interval is defined to be significantly shorter than the average R-R interval if: a) the considered R-R interval is shorter than the average R-R interval, and b) if the second deviation is greater than the increased first deviation. The considered R-R interval is defined to be significantly longer than the average R-R interval if: a) the considered R-R interval is longer than the average R-R interval, and b) if the second deviation is greater than the increased first deviation. Thus, this embodiment does not rely on a comparison of consecutive R-R intervals nor on a calculation of a ratio between different R-R intervals. Rather, the reference point for deciding whether a considered R-R interval is a particularly long or a particularly short R-R interval is the average R-R interval. Thereby, the factor being greater than one determines a safety margin applied for classifying individual R-R intervals as ectopic R-R intervals. The bigger the factor, the less likely is the classification of individual R-R intervals as ectopic R-R intervals. Since this embodiment does not rely on or necessitates a static threshold, but rather enables use of a dynamic average R-R interval being updated continuously (due to use of always the actual latest R-R intervals for calculating the average R-R interval), the ectopy detection is continuously adapted to possible changes in the cardiac rhythm of the considered patient.

In an embodiment, the factor is a number between 1.05 and 5, in particular between 1.1 and 4.9, in particular between 1.2 and 4.8, in particular between 1.3 and 4.7, in particular between 1.4 and 4.6, in particular between 1.5 and 4.5, in particular between 1.6 and 4.4, in particular between 1.7 and 4.3, in particular between 1.8 and 4.2, in particular between 1.9 and 4.1, in particular between 2.0 and 4.0, in particular between 2.1 and 3.9, in particular between 2.2 and 3.8, in particular between 2.3 and 3.7, in particular between 2.4 and 3.6, in particular between 2.5 and 3.5, in particular between 2.6 and 3.4, in particular between 2.7 and 3.3, in particular between 2.8 and 3.2, in particular between 2.9 and 3.1, in particular between 3.0 and 3.1. A factor lying in a range between 1.5 and 2.5 is particularly appropriate for carrying out this embodiment.

In an embodiment, the first deviation is recalculated if the average R-R interval is updated. As outlined above, the R-R interval is typically regularly updated with each new incoming cardiac signal delimiting a further R-R interval. Thus, this recalculation of the first deviation is, in an embodiment, carried out continuously.

In an embodiment, the average R-R interval is recalculated with each newly detected R-R interval if this newly detected R-R interval is not discarded from calculating the average R-R interval. Thus, while R-R intervals comprising an ectopic cardiac signal are also in this embodiment discarded from calculating the average R-R interval, this embodiment guarantees for continuous update of the average R-R interval so that the average R-R interval is adapted to any changes in the cardiac rhythm.

As explained above, the first and the second of the two consecutive R-R intervals are discarded from calculating the average R-R interval in case that an ectopic signal has been recognized in either of the first or the second of the two consecutive R-R intervals. In an embodiment, also two R-R intervals preceding the first of the two consecutive R-R intervals are discarded from detection of the presence of atrial fibrillation.

In an embodiment, also the oldest R-R interval available for calculating the average R-R interval is discarded from detection of the presence of atrial fibrillation.

In an embodiment, the first of the two consecutive R-R intervals is considered to be significantly shorter than the average R-R interval if the first of the two consecutive R-R intervals is at least 5%, in particular between 5% and 100%, in particular between 10% and 90%, in particular between 20% and 80%, in particular between 30% and 70%, in particular between 40% and 60%, in particular 50% shorter than the average R-R interval.

In an embodiment, the second of the two consecutive R-R intervals is considered to be significantly longer than the average R-R interval if the second of the two consecutive R-R intervals is at least 5%, in particular between 5% and 500%, in particular between 10% and 490%, in particular between 20% and 480%, in particular between 30% and 470%, in particular between 40% and 460%, in particular between 50% and 450%, in particular between 60% and 440%, in particular between 70% and 430%, in particular between 80% and 420%, in particular between 90% and 410%, in particular between 100% and 400%, in particular between 110% and 390%, in particular between 120% and 380%, in particular between 130% and 370%, in particular between 140% and 360%, in particular between 150% and 350%, in particular between 160% and 340%, in particular between 170% and 330%, in particular between 180% and 320%, in particular between 190% and 310%, in particular between 200% and 300%, in particular between 210% and 290%, in particular between 220% and 280%, in particular between 230% and 270%, in particular between 240% and 260%, in particular between 250% and 260%, longer than the average R-R interval.

In an embodiment, a number of detected ectopic signals is counted. This counted number can then be used in other methods for making specific decisions, e.g., whether or not certain sections of the cardiac rhythm are to be used for the detection of atrial fibrillation or whether certain sections of an electrocardiogram are to be discarded.

In an aspect, the present invention relates to an implantable medical device for detecting an electrical signal of a human or animal heart. This implantable medical device comprises a processor, a memory unit, and a detection unit configured to detect electrical signals of a human or animal heart. In this context, the memory unit comprises a computer readable program that causes the processor to perform the following steps when executed on the processor.

In a first step, consecutive R-R intervals are detected or identified in an electrocardiogram.

Afterwards, an average R-R interval is calculated for a determinable number of latest consecutive R-R intervals.

Afterwards, the signal is recognized or identified as ectopic signal if the signal belongs to at least one of two consecutive R-R intervals and if the two conditions explained in the following are fulfilled.

The following two conditions need to be met for recognizing a signal as an ectopic signal. First, the first of the two consecutive R-R intervals needs to be at least 5% shorter than the average R-R interval. Second, a second of the two consecutive R-R intervals needs to be at least 5% longer than the average R-R interval. In this context, the second R-R interval occurs later than the first R-R interval in the electrocardiogram.

In order to achieve a particularly reliable recognition of ectopic signals, the first and the second of the two consecutive R-R intervals are discarded from calculating the average R-R interval if the signal is recognized as an ectopic signal. Avoiding using of the two consecutive R-R intervals (also referred to as ectopic R-R intervals) for calculating the average R-R interval results in a more reliable average R-R interval and thus in a more reliable detection of subsequent ectopic signals.

In an aspect, the present invention relates to a computer program product, in particular to a non-transitory computer program product, comprising computer readable code that causes a processor to perform the following steps when executed on the processor.

In a first step, consecutive R-R intervals are detected or identified in an electrocardiogram by a detection unit.

Afterwards, an average R-R interval is calculated for a determinable number of latest consecutive R-R intervals.

Afterwards, the signal is recognized or identified as ectopic signal if the signal belongs to at least one of two consecutive R-R intervals and if the two conditions explained in the following are fulfilled.

The following two conditions need to be met for recognizing a signal as an ectopic signal. First, the first of the two consecutive R-R intervals needs to be significantly shorter than the average R-R interval. Second, a second of the two consecutive R-R intervals needs to be significantly longer than the average R-R interval. In this context, the second R-R interval occurs later than the first R-R interval in the electrocardiogram.

Finally, the first and the second of the two consecutive R-R intervals are discarded from calculating the average R-R interval if the signal is recognized as an ectopic signal.

In an aspect, the present invention relates to a method for detecting atrial fibrillation of a human or animal heart, wherein the method comprises applying an atrial fibrillation detection algorithm (such as a common atrial fibrillation detection algorithm generally known per se to a person skilled in the art) and a method for detecting an ectopic signal in an electrocardiogram according to the preceding explanations. Then, any atrial fibrillation can be much more reliably detected since ectopic signals are more reliably detected than according to prior art methods.

In an embodiment, the method for detecting an ectopic signal in an electrocardiogram is only carried out for a programmable time period after beginning of the atrial fibrillation detection algorithm. In an embodiment, the programmable time period lies in a range between 30 seconds and 6 minutes, in particular between 1 minute and 5 minutes, in particular between 1.5 and 4.5 minutes, in particular between 2 and 4 minutes, in particular between 2.5 and 3.5 minutes, in particular 3 minutes.

According to an embodiment, the method for detecting an ectopic signal in an electrocardiogram is only carried out or for a programmable time period after the detection of an atrial fibrillation episode.

In an embodiment, a number of detected ectopic signals is counted, wherein an observed cardiac rhythm is considered being a rhythm devoid of atrial fibrillation if the number of detected ectopic signals exceeds a threshold. As an example, the observed cardiac rhythm is considered devoid of atrial fibrillation if 2 to 10 ectopic signals are detected within 10 to 25 intervals. In particular, the observed cardiac rhythm is considered devoid of atrial fibrillation if 4 ectopic signals are detected within 16 intervals.

According to an embodiment of the present invention, an observed cardiac rhythm is considered being a rhythm devoid of atrial fibrillation if the number of detected ectopic signals plus a number of detected noise events exceeds a threshold.

All embodiments and variants of the different methods described herein can be combined in any desired way and can be transferred individually or in any arbitrary combination to the respective other method, to the computer program product and to the implantable medical device. Likewise, all variants and embodiments of the described implantable medical device can be combined in any desired way and can be transferred individually or in any arbitrary combination to either of the methods or to the described computer program product. Finally, all variants and embodiments described with respect to the computer program product can be combined in any desired way and can be transferred either individually or in any arbitrary combination to any of the described methods and to the implantable medical device.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

Further details of aspects of the present invention will be explained with respect to exemplary embodiments and accompanying Figures. In the Figures:

FIG. 1 is an exemplary section of an electrocardiogram comprising a premature ventricular contraction (PVC);

FIG. 2 shows an example of a buffer of intervals used by an algorithm for detecting atrial fibrillation;

FIG. 3 shows a schematic flowchart of an exemplary embodiment of a method for detecting an ectopic signal in an electrocardiogram;

FIG. 4 shows a schematic flowchart of a method of atrial fibrillation detection with integrated ectopy detection;

FIG. 5 shows a schematic flowchart of a method of ectopy detection threshold calculations;

FIG. 6 shows a first exemplary electrocardiogram and a first ectopy detection result plot; and

FIG. 7 shows a second exemplary electrocardiogram and a second ectopy detection result plot.

### DETAILED DESCRIPTION

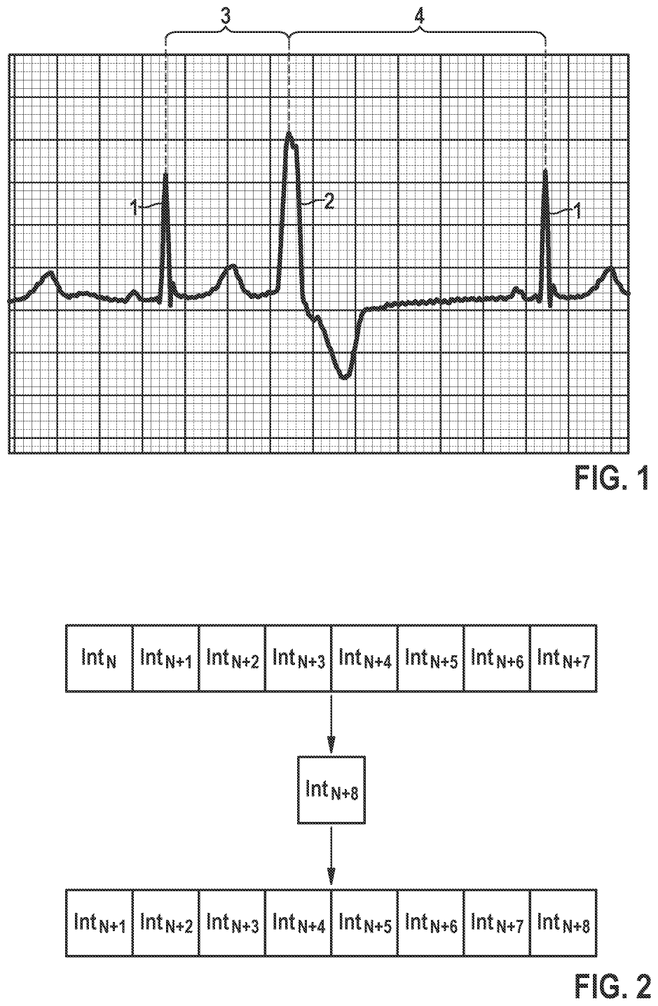

FIG. 1 shows an exemplary section of an electrocardiogram. Two regular ventricular depolarizations 1 can be seen in the first third and in the last third of this electrocardiogram. In between, a premature ventricular contraction (PVC) 2 is visible. A first R-R interval 3 extends from the first normal ventricular depolarization 1 to the PVC 2. This first R-R interval 3 is a short interval. A second R-R interval 4 extends from the PVC 2 to the second regular ventricular depolarization 1. This second R-R interval 4 is a compensatory pause interval being longer than usual R-R intervals.

The presence of the short R-R interval 3 followed by the long R-R interval 4 is indicative for an ectopy caused by an ectopic beat in form of the PVC 2.

The first normal ventricular depolarization 1 belongs both to the short R-R interval 3 as well as to the preceding R-R interval. The PVC 2 belongs both to the short R-R interval 3 as well as to the long R-R interval 4. The second normal ventricular depolarization 1 belongs both to the long R-R interval 4 as well as to the next succeeding R-R interval.

FIG. 2 generally shows how individual R-R intervals of an electrocardiogram are stored in a buffer of an implantable medical device for sensing or detecting electrical signals of a human or animal heart. This general proceeding does not only apply for methods for detecting an ectopic signal in an electrocardiogram, but more broadly also for other signal processing algorithms like atrial fibrillation (AF) detection algorithms.

The top of FIG. 2 shows a row representing a buffer of stored R-R intervals, wherein Int_{N} is the oldest interval and the intervals Int_{N+1}, Int_{N+2}, Int_{N+3}, Int_{N+4}, Int_{N+5}, Int_{N+6} are progressively newer. Int_{N+7} is the newest interval. This row of intervals can be considered as a number of latest consecutive R-R intervals, wherein the number is—in the exemplary embodiment depicted in FIG. 2—eight.

If a novel R-R interval Int_{N+8} is measured, this novel interval is added to the row of intervals, wherein the oldest interval Int_{N} is deleted. Consequently, now Int_{N+1} is the oldest interval, wherein Int_{N+8} is the newest interval. Still then, the buffer comprises eight latest consecutive R-R intervals Int_{N+1} to Int_{N+8}.

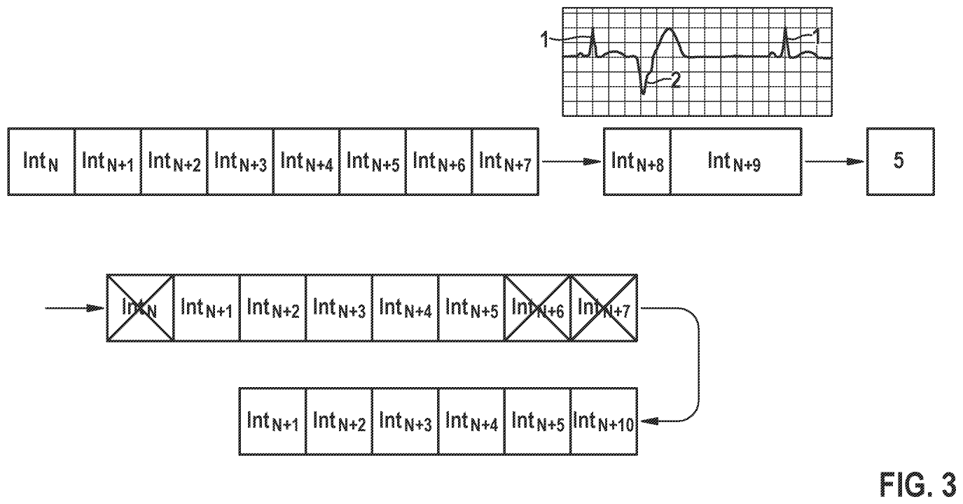

FIG. 3 exemplarily shows an embodiment of a method for detecting an ectopic signal in an electrocardiogram. Starting with eight R-R intervals Int_{N} to Int_{N+7} (as explained with respect to FIG. 2), two further R-R intervals Int_{N+8} and Int_{N+9} are detected. The R-R interval Int_{N+8} is a short R-R interval reaching from a regular ventricular depolarization 1 to a premature ventricular contraction (PVC) 2. Here and in all following Figures, the same numeral references for the same or similar elements are used.

The R-R interval Int_{N+9} is a longer interval reaching from the PVC 2 to a second regular ventricular depolarization 1. Consequently, an R-R interval being shorter than the average R-R intervals and a consecutive R-R interval being longer than the average R-R interval would be added to the row of R-R intervals Int_{N} to Int_{N+7}. This specific pattern of the short R-R interval Int_{N+8} and the long interval Int_{N+9} results in a detection 5 of an ectopic signal in the electrocardiogram. As a consequence, these two consecutive R-R intervals Int_{N+8} and Int_{N+9} are discarded from a buffer for detection of the presence of atrial fibrillation. Furthermore, the two intervals Int_{N+6} and Int_{N+7} preceding these two consecutive intervals are also discarded from the buffer for detection of the presence of atrial fibrillation. Finally, the oldest R-R interval Int_{N} is also discarded from the buffer for detection of the presence of atrial fibrillation.

After having detected the next regular R-R interval Int_{N+10}, a reduced row of R-R intervals comprising Int_{N+1}, Int_{N+2}, Int_{N+3}, Int_{N+4}, Int_{N+5}, and Int_{N+10} results. Then, only these intervals are used for calculating the average R-R interval. Int_{N+5} and Int_{N+10} are now considered and treated like consecutive R-R intervals in the interval buffer.

Discarding the first short R-R interval Int_{N+8} and the second long R-R interval Int_{N+9} as well as the two R-R intervals Int_{N+6} and Int_{N+7} preceding the two short and long R-R intervals results in a significant more accurate calculation of the average R-R interval. Discarding also the oldest R-R interval Int_{N} from the calculation of the average R-R interval further enhances reliability, but is, in other embodiments of the method for detecting an ectopic signal in an electrocardiogram—not implemented.

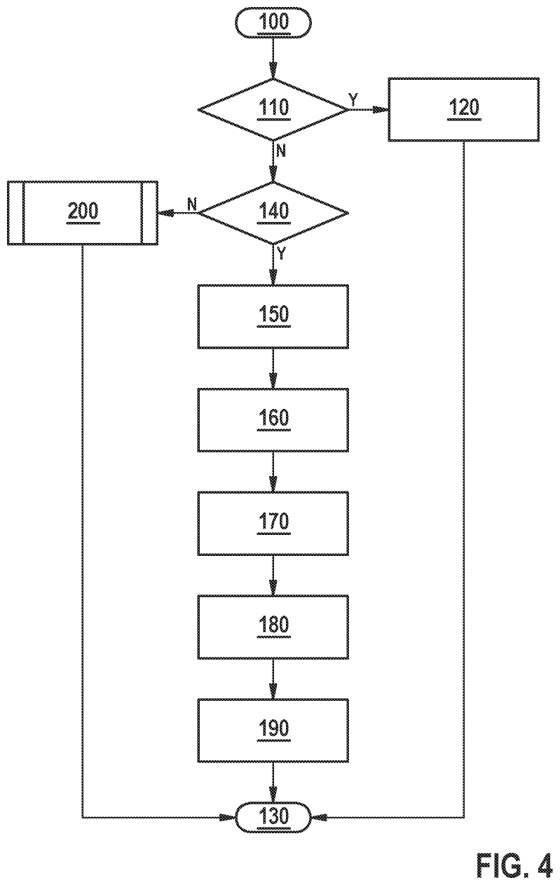

FIG. 4 shows a schematic flowchart of a method of atrial fibrillation detection with integrated ectopy detection.

A QRS complex is detected in an electrocardiogram in a first method step 100 prior to detection of atrial fibrillation. Then, the method for detecting an ectopic signal in an electrocardiogram checks in a second step 110 if a parameter called “IgnoreOneInterval” is set to true. If this is the case (Y), this parameter is set to false in further step 120. Afterwards, the method for atrial fibrillation detection is continued in a regular method step 130. The parameter “IgnoreOneInterval” is said to true if an ectopic signal has been detected in the preceding method step, namely by the presence of an R-R interval being shorter than a short threshold followed by an R-R interval being longer than a long threshold. In such a case, normal atrial fibrillation detection 130 is to be applied rather than checking if another ectopic signal is to be detected in the electrocardiogram.

If, however, the parameter “IgnoreOneInterval” is not set to true (N), ectopy detection takes place. In a further method step 140, it is checked if the older of two consecutive R-R intervals was shorter than or equal to a short threshold and if at the same time the newer of two consecutive R-R intervals was longer than or equal to a long threshold. If this is true (Y), the result of method step 140 is a detected ectopy 150. Following ectopy detection, the ventricular signal that concludes a short interval is changed to a noise event (ventricular noise) in the next method step 160 for the atrial fibrillation algorithm. In addition, the R-R interval count is decremented twice in the next method step 170. Afterwards, the two newest intervals are removed from the R-R interval buffer in the next method step 180. According to an embodiment of the present invention, the ectopy detection is considered as ventricular noise by the atrial fibrillation detection algorithm, these ectopy detections lead to atrial fibrillation detection termination during the noise window that starts at the atrial fibrillation detection. According to an alternative embodiment, an ectopy detection leads to termination of an atrial fibrillation detection during the ectopy window, regardless of whether the noise window is simultaneously ongoing. When the interval buffer is full, then the noise count that leads to atrial fibrillation detection termination is cleared. Finally, in the next method step 190, the parameter “IgnoreOneInterval” is set to true so that the next interval is not allowed to change the ectopy detection thresholds. In any case, now a regular atrial fibrillation detection algorithm 130 is carried out.

If in the method step 140 the older R-R interval was not shorter than the short threshold and/or the newer R-R interval was not longer than the long threshold (N), then an update 200 of the short and long thresholds for ectopy detection is carried out. After this update 200, a regular atrial fibrillation detection algorithm 130 is carried out.

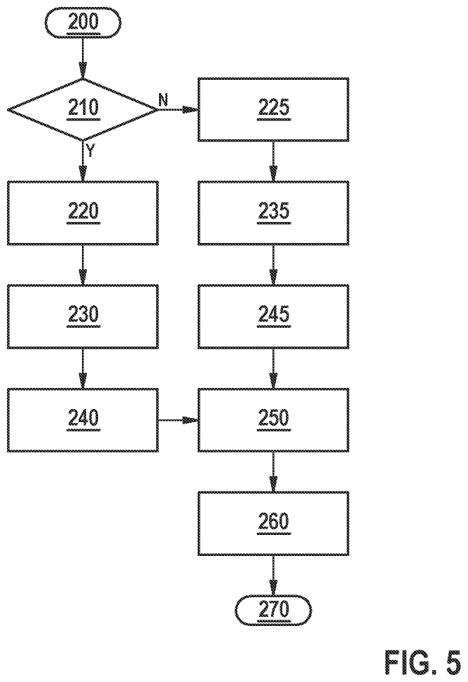

FIG. 5 shows an exemplary embodiment of the update process 200 of FIG. 4.

In a first step 210 of this update process, it is checked if the parameter “BuffLen” is equal to 1. This parameter represents the size of the average used for the mean R-R interval and the mean deviation from the average R-R interval (this deviation parameter is also called “intDeltaX”). BuffLen is an integer that will never be smaller than 1.

If BuffLen is 1 (Y), then the average R-R interval is the previous R-R interval, and the mean absolute deviation from the average is the difference between the old R-R interval and the previous R-R interval (intDeltaX=|(aveInt−oldInt)|) (step 220).

The variable “intDelta” is calculated as the maximum of “intDeltaX” times “DeltaLim” and the average interval times the parameter “LoIntLim” (intDelta=MAX(intDeltaX* DeltaLim, aveInt*LoIntLim) (step 230).

Finally, the actual average R-R interval (aveInt) is equal to the previous old R-R interval (oldInt) (aveInt=oldInt) (step 240). This method then continues with method step 250 (see below).

If BuffLen is greater than 1 (N), the average interval is updated with an exponential moving average (EMA) filter (aveInt=aveInt*(BuffLen−1)/BuffLen+(1/BuffLen)* oldInt) (step 225). In addition, the average interval difference from the mean (intDeltaX) is updated using an EMA filter (intDeltaX=intDeltaX*(BuffLen−1)/BuffLen+(1/BuffLen)*|(aveInt−oldInt)|) (step 235). Once intDeltaX is calculated, intDelta is updated. The variable “intDelta” is also in this branch of the method the maximum of “intDeltaX” times “DeltaLim” and the average interval times the parameter “LoIntLim” (intDelta=MAX(intDeltaX*DeltaLim, aveInt*LoIntLim) (step 245).

The variable “intDelta” is then subtracted from the variable “aveInt” to create the short threshold ectopy detection limit for the next ectopy test (short threshold=aveInt−intDelta) (step 250). Likewise, the variable “intDelta” is then added to the variable “aveInt” to create the long threshold ectopy detection limit for the next ectopy test (long threshold=aveInt+intDelta) (step 260).

Finally, an exit 270 of the update process is reached.

An aspect of the presently described method for detecting an ectopic signal in an electrocardiogram is that the ectopy detection is less sensitive as the beat-to-beat interval variability increases. Thus, it is harder to falsely detect ectopies during atrial fibrillation or during other irregular cardiac rhythms having a large beat-to-beat interval variability.

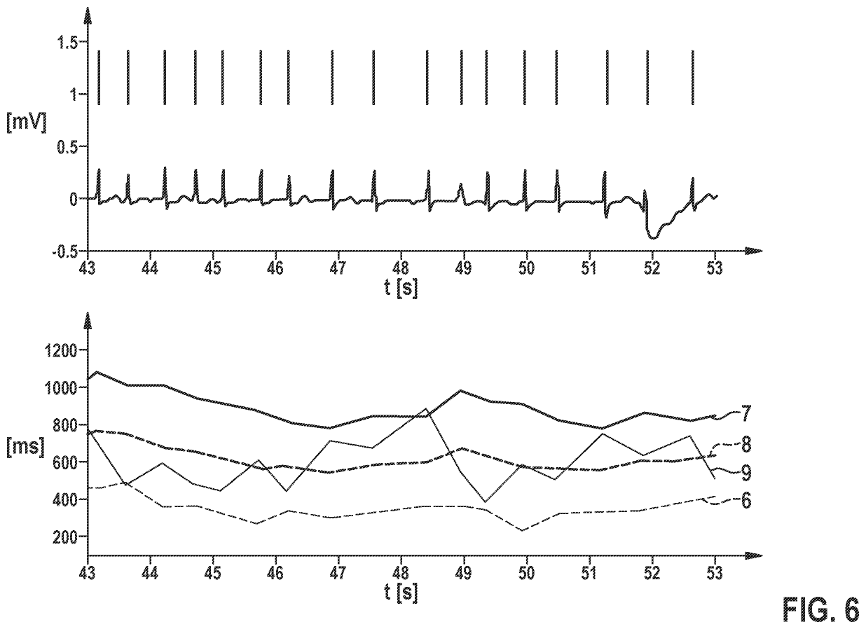

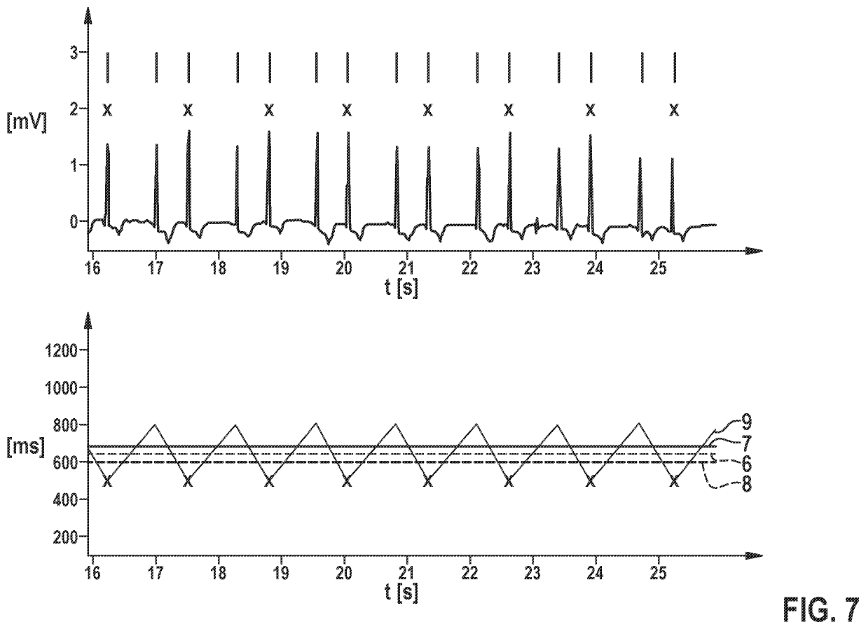

FIGS. 6 and 7 exemplarily show the behavior of the novel method for detecting an ectopic signal in an electrocardiogram making reference to an electrocardiogram and its evaluation as measured by an implantable medical device for detecting electrical signals of a human or animal heart.

Both in FIG. 6 and in FIG. 7, the upper panel shows an electrocardiogram with signals in mV over the time in seconds. Ventricular signals in the electrocardiogram are marked with vertical lines in the top of the top panels of FIGS. 6 and 7. The bottom plots of FIGS. 6 and 7 show an ectopy algorithm average interval 6, an upper ectopy detection threshold 7, a lower ectopy detection threshold 8, and actual R-R intervals 9. The ectopy algorithm average interval 6 forms the basis for calculating the upper ectopy detection threshold 7 (longer threshold) and the lower ectopy detection threshold 8 (short threshold).

In case of atrial fibrillation (cf. FIG. 6), the actual R-R intervals 9 are highly volatile. Consequently, a wide corridor is formed by the upper ectopy detection threshold 7 and the lower ectopy detection threshold 8. Consequently, no ectopy is detected during this cardiac rhythm state.

In contrast, FIG. 7 shows a typical sinus rhythm of a heart resulting in a narrow corridor between the upper ectopy detection threshold 7 and the lower ectopy detection threshold 8. As a result, a plurality of detected cardiac signals is classified as ectopic signals, each resulting from an ectopic beat (marked with an X in the upper panel of FIG. 7). In this case, due to a lack of continuous updates of the short and long thresholds, the depicted algorithm is prone to false detections of ectopic beats. Thus, using the continuous update of the short and long thresholds for detecting ectopic signals according to the presently described invention, the method is less prone to falsely detect ectopic signals in case of atrial fibrillation than methods known from prior art. At the same time, ectopic signals can be reliably detected during times of stable cardiac sinus rhythm.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A method for detecting an ectopic signal in an electrocardiogram, the ectopic signal being caused by an ectopic cardiac beat, the method comprising the following steps that are performed by a processor of an implantable medical device when a computer-readable program comprised in a memory of the implantable medical device is executed on the processor:
detecting, with a detection unit of the implantable medical device, consecutive R-R intervals in an electrocardiogram;
calculating an average R-R interval for a determinable number of latest R-R intervals;
recognizing a signal as ectopic signal if the signal belongs to at least one of two consecutive R-R intervals, wherein
i) a first of the two consecutive R-R intervals is at least 5% shorter than the average R-R interval; and
ii) a second of the two consecutive R-R intervals is at least 5% longer than the average R-R interval, wherein the second R-R interval occurs later than the first R-R interval;
wherein the first and the second of the two consecutive R-R intervals are discarded from calculating the average R-R interval, if the signal is recognized as ectopic signal.

2. The method according to claim 1, wherein the method further comprises the following steps: calculating a first deviation, the first deviation being an average absolute deviation of the consecutive R-R intervals of the determinable number of latest consecutive R-R intervals used for calculating the average R-R interval from the average R-R interval; multiplying the first deviation with a factor being greater than 1 to obtain an increased first deviation; calculating a second deviation, the second deviation being an absolute deviation of a successive R-R interval from the average R-R interval; defining the R-R interval as significantly shorter than the average R-R interval if the R-R interval is shorter than the average R-R interval and if the second deviation is greater than the increased first deviation, or defining the R-R interval as significantly longer than the average R-R interval if the R-R interval is longer than the average R-R interval and if the second deviation is greater than the increased first deviation.

3. The method according to claim 2, wherein the factor is a number between 1.05 and 5.

4. The method according to claim 2, wherein the first deviation is recalculated if the average R-R interval is updated.

5. The method according to claim 1, wherein the average R-R interval is recalculated with each newly detected R-R interval if this newly detected R-R interval is not discarded from calculating the average R-R interval.

6. The method according to claim 1, wherein, if the first and the second of the two consecutive R-R intervals are discarded from calculating the average R-R interval.

7. The method according to claim 1, wherein the first of the two consecutive R-R intervals is considered to be significantly shorter than the average R-R interval if the first of the two consecutive R-R intervals is at least 5% shorter than the average R-R interval.

8. The method according to claim 1, wherein the second of the two consecutive R-R intervals is considered to be significantly longer than the average R-R interval if the second of the two consecutive R-R intervals is at least 5% longer than the average R-R interval.

9. The method according to claim 1, wherein a number of detected ectopic signals is counted.

10. An implantable medical device for detecting an electrical signal of a human or animal heart, the implantable medical device comprising a processor, a memory unit, and a detection unit configured to detect an electrical signal of a human or animal heart, wherein the memory unit comprises a computer-readable program that causes the processor to perform the following steps when executed on the processor:
detecting, with the detection unit, consecutive R-R intervals in an electrocardiogram;
calculating an average R-R interval for a determinable number of latest consecutive R-R intervals;
recognizing a signal as ectopic signal if the signal belongs to at least one of two consecutive R-R intervals, wherein
i) a first of the two consecutive R-R intervals is at least 5% shorter than the average R-R interval; and
ii) a second of the two consecutive R-R intervals is at least 5% longer than the average R-R interval, wherein the second R-R interval occurs later than the first R-R interval;
wherein the first and the second of the two consecutive R-R intervals are discarded from calculating the average R-R interval.

11. A method for detecting atrial fibrillation of a human or animal heart, the method comprising applying an atrial fibrillation detection algorithm and the method for detecting an ectopic signal in an electrocardiogram according to claim 1.

12. The method according to claim 11, wherein the method for detecting an ectopic signal in an electrocardiogram is only carried out for a programmable time period after a beginning of the atrial fibrillation detection algorithm, or for a programmable time period after the detection of an atrial fibrillation episode.

13. The method according to claim 11, wherein a number of detected ectopic signals is counted and wherein an observed cardiac rhythm is considered being a rhythm devoid of atrial fibrillation if the number of detected ectopic signals exceeds a threshold.

14. The method according to claim 12, wherein an observed cardiac rhythm is considered being a rhythm devoid of atrial fibrillation if the number of detected ectopic signals plus a number of detected noise events exceeds a threshold.
